SEQUENCE LISTING (2 sequences)

SEQ ID NO: 1 - protein, 305 aa, Helicobacter pylori
MILTAMQDAIGRTPIFKFTRKDYPIPLKSAIYAKLEHLNPGGSVKDRLGQYLIKEAFRTHKITSTTTIIEPTAGNTGIALALVAIKHHLKTIFVVPEKFSVEKQQIMRALGALVINTPTSEGISGAIKKSKELAESIPDSYLPLQFENPDNPAAYYHTLAPEIVKELGTNFTSFVAGIGSGGTFAGTAKYLKERIPNIRLIGVEPEGSILNGGEPGPHEIEGIGVEFIPPFFANLDIDGFETISDEEGFSYTRKLAKKNGLLVGSSSGAAFAAALKEVQRLPEGSQVLTIFPDMADRYLSKGIYS

SEQ ID NO: 2 - protein, 378 aa, Helicobacter pylori
MQTKLIHGGISEDATTGAVSVPIYQASTYRQDAIGRHKGYEYSRSGNPTRFALEELIADLEGGVKGFAFASGLAGIHAVFSLLQSGDHVLLGDDVYGGTFRLFNKVLVKNGLSCTIIDTSDISQIKKAIKPNTKALYLETPSNPLLKITDLAQCASVAKDHGLLTIVDNTFATPYCQNPLLLGADIVAHSGTKYLGGHSDVVAGLVTTNNEALAQEIAFFQNAIGGVLGPQDSWLLQRGIKTLGLRMEAHQKNALCVAEFLEKHPKVERVYYPGLPTHPNHELAKAQMRGFSGMLSFTLKNDSEAALFVESLKLFILGESLGGVESLVGIPALMTHACIPKEQREAAGIRDGLVRLSVGIEHEQDLLEDLEQAFAKIS

(19) United States
(12) Patent Application Publication
(10) Pub. No.: US 2014/0045193 A1
(43) Pub. Date: Feb. 13, 2014

(54) METHIONINE METABOLITES PREDICT AGGRESSIVE CANCER PROGRESSION

(71) Applicant: Cedars-Sinai Medical Center, Los Angeles, CA (US)

(72) Inventors: Neil A. Bhowmick, Beverly Hills, CA (US); Diptiman Choudhury, Los Angeles, CA (US)

(73) Assignee: Cedars-Sinai Medical Center, Los Angeles, CA (US)

(21) Appl. No.: 13/963,922

(22) Filed: Aug. 9, 2013

Related U.S. Application Data
(60) Provisional application No. 61/682,155, filed on Aug. 10, 2012.

(51) Int. Cl. G01N 33/68

(52) U.S. Cl. CPC G01N 33/6893
USPC 435/7.8

(57) ABSTRACT

The invention relates to the use of enzymes and nanorods to detect cysteine level in a patient sample and relates to the use of the detected cysteine level to predict cancer recurrence in the patient. The invention is directed to systems and methods of detecting cysteine level in a sample from a subject, wherein the systems or methods can further comprise measuring at least one additional parameter, such as PSA level, Gleason score and clinical stage. The invention is directed to systems and methods of predicting the probability of a recurrence of a cancer in a subject, wherein the systems or methods can further comprise measuring at least one additional parameter, such as PSA level, Gleason score and clinical stage. This invention is directed to systems and methods of predicting the probability of a recurrence of a cancer in a subject and treating the subject, wherein the systems or methods can further comprise measuring at least one additional parameter, such as PSA level, Gleason score and clinical stage.

### FIELD OF INVENTION

This invention relates to the fields of urology, oncology and pathology. More specifically, this invention relates to systems and methods for predicting the probability of prostate cancer recurrence in a subject before, during, or after cancer treatment. This invention also relates to systems and methods for detecting a cysteine level in a sample from a subject.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Prostate cancer remains the most common non-cutaneous solid malignancy in the United States, and the second leading cause of cancer specific death in men. Nevertheless, it has become increasingly clear that not all men who are diagnosed with prostate cancer require intervention [1]. Yet, many men that receive surgical or radiation-based primary treatment develop recurrent disease. Prior to surgical intervention, serum PSA, biopsy Gleason grade, and clinical stage help determine if patients are likely to recur versus those that may remain localized and possibly remain clinically inconsequential. Various approaches in improving the role of PSA in early prostate cancer detection have been tested, but their benefit to overall survival is yet to be proven [2,3]. Ultimately, there is a subgroup of men without conventional negative factors harboring high risk, aggressive disease and are even at elevated risk of early recurrence after attempted definitive local therapy [4,5,6]. The ongoing challenge facing clinicians is how to identify this cohort of men at high risk, from the larger cohort of men who are likely harboring more indolent disease [7]. New markers of aggressive disease are therefore needed for an informed clinical decision.

A previous study identified sarcosine (N-methylglycine) as a product of methionine catabolism that is elevated in the urine of patients with metastatic prostate disease [8]. Sarcosine levels were higher in tissues from localized prostate cancer than in normal tissue, and even higher in metastatic prostate tissue. Urinary sarcosine was thus suggested as a possible marker for metastatic prostate cancer. The enzyme, Glycine N-methyltransferase (GNMT) is the primary source of sarcosine in liver, where it accounts for about 1% of the soluble protein [9]. Individuals with defective sarcosine dehydrogenase have sarcosinemia, but show no distinctive phenotype [10]. However, a reported causative role for sarcosine in prostate cancer metastasis [8] suggests therapeutic targeting of its metabolic pathway to be useful.

Nevertheless, the current markers only suggest the presence or absence of cancer and they are not shown to have any predicative value. As such, there is still great need of markers, methods and systems that can predict the probability of recurrent cancer prior to surgical intervention or any cancer treatment. In this invention, we demonstrate that the cysteine level in urine or serum is a predictive marker for cancer recurrence. We provide systems and methods of predicting the probability of cancer recurrence based on the cysteine level, and we provide systems and methods of detecting the cysteine level in urine or serum using a combination of enzymes and nanorods.

### SUMMARY OF THE INVENTION

The following embodiments and aspects thereof are described and illustrated in conjunction with systems, compositions and methods which are meant to be exemplary and illustrative, not limiting in scope.

Various embodiments of the present invention provide for a system for detecting a cysteine level in a sample from a subject. The system can comprise cystathionine synthase, cystathionine lyase, and nanorods. The system can further comprise a PSA test, clinical stage, biopsy Gleason score, pathologic Gleason score, pathologic stage, surgical margin status, lymph node involvement, or seminal vesicle involvement, or a combination thereof.

Various embodiments of the present invention provide for a method of detecting a cysteine level in a sample from a subject. The method comprises: obtaining a sample from the subject; processing the sample with cystathionine synthase and cystathionine lyase; mixing the processed sample with nanorods; measuring a change of absorption spectrum of the nanorods; and detecting the cysteine level based upon the change of absorption spectrum.

Various embodiments of the present invention provide for a system for predicting the probability of a recurrence of a cancer in a subject. The system comprises an isolated sample from the subject, cystathionine synthase, cystathionine lyase, and nanrods. The system can further comprise a PSA test, clinical stage, biopsy Gleason score, pathologic Gleason score, pathologic stage, surgical margin status, lymph node involvement, or seminal vesicle involvement, or a combination thereof.

Various embodiments of the present invention provide for a method of predicting the probability of a recurrence of a cancer in a subject. The method comprises: obtaining a sample from the subject; processing the sample with cystathionine synthase and cystathionine lyase; subjecting the processed sample to an assay to detect cysteine level, wherein the assay comprises nanorods; and predicting an increased probability of the recurrence of the cancer in the subject when the cysteine level in the subject is detected to be higher than in non-recurrent subjects. The method can further comprise active surveillance, prostatectomy, chemotherapy, immunotherapy, hormone therapy, radiation therapy, focal therapy, systemic therapy, high frequency ultrasound (HIFU), cryo therapy, brachytherapy, or a combination thereof.

Various embodiments of the present invention provide for a method of predicting the probability of a recurrence of a cancer in a subject. The method comprises: obtaining a sample from the subject; processing the sample with cystathionine synthase and cystathionine lyase; subjecting the processed sample to an assay to detect cysteine level, wherein the assay comprises nanorods; assessing at least one additional parameter; and predicting an increased probability of the recurrence of the cancer in the subject when the cysteine level in the subject is detected to be higher than in non-recurrent subjects and/or when the additional parameter in the subject is detected to be higher or lower than in non-recurrent subjects. The method can further comprise active surveillance, prostatectomy, chemotherapy, immunotherapy, hormone therapy, radiation therapy, focal therapy, systemic therapy, high frequency ultrasound (HIFU), cryo therapy, brachytherapy, or a combination thereof.

Various embodiments of the present invention provide for a method of predicting the probability of a recurrence of a cancer in a subject. The method comprises: obtaining a sample from the subject; processing the sample with cystathionine synthase and cystathionine lyase; subjecting the processed sample to an assay to detect cysteine level; and predicting an increased probability of the recurrence of the cancer in the subject when the cysteine level in the subject is detected to be higher than in non-recurrent subjects. The method can further comprise active surveillance, prostatectomy, chemotherapy, immunotherapy, hormone therapy, radiation therapy, focal therapy, systemic therapy, high frequency ultrasound (HIFU), cryo therapy, brachytherapy, or a combination thereof.

In various embodiments, the recurrence can be biochemical recurrence. In various embodiments, the cancer is prostate cancer, colon cancer, breast cancer, lung cancer, renal cancer, or bladder cancer. In various embodiments, the subject can be human, monkey, ape, dog, cat, cow, horse, goat, pig, rabbit, mouse, or rat. In various embodiments, the sample can be obtained before, during, or after cancer treatment. In various embodiments, the sample can be serum, urine, blood, plasma, saliva, semen, lymph, or a combination thereof. In various embodiments, the sample is urine and the urine cysteine level in the subject is above about 210, 220, or 230 nanomoles of cysteine per milligram creatinine. In various embodiments, the sample is serum and the serum cysteine level in the subject is above about 400, 410, or 420 μM of cysteine. In various embodiments, the nanorods can be made of gold, selenium, cadmium, copper, or a combination thereof. In various embodiments, the cystathionine synthase comprises a polypeptide having a sequence as set forth in SEQ ID NO: 1. In various embodiments, the cystathionine lyase comprises a polypeptide having a sequence as set forth in SEQ ID NO: 2. In some embodiments, the PSA test is a test of PSA velocity and/or total PSA level. PSA velocity means the rate at which PSA level rises over time.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts Kaplan-Meier plots indicating univariate predictive values of the recurrence-free survival based on pre-surgical serum in accordance with various embodiments of the present invention. The patients were separated into two groups, divided at median tissue level for (A) PSA, (B) homocysteine, (C) cystathionine, and (D) cysteine as significantly associated with time to recurrence (Table 5). Those subjects above the median expression level were termed upper half, whereas those below the median were termed lower half. The recurrence-free survival probabilities were estimated by the Kaplan-Meier method and the differences were tested using the log-rank test. Each of the dichotomous serum markers supported statistically significant differences in biochemical recurrence-free survival.

FIGS. 2A, B, and C depict Receiver Operator Curve (ROC) for a statistical model that can be used to predict recurrence of prostate cancer based on serum derived variables in accordance with various embodiments of the present invention. Serum PSA is compared to the added value of serum (A) homocysteine, (B) cystathionine, and (C) cysteine. In the ROC curve the probability with greater Area Under the Curve (AUC) support increased specificity and sensitivity over random guess, represented by the dotted diagonal line.

FIG. 3 depicts methionine metabolism. Methionine is first converted to SAM, the donor of methyl groups in all but one methyltransferase reaction. SAM may transfer the methyl group to a variety of compounds, X, by a group of specific enzymes to yield the methylated compounds, CH3-X (eg. methylated lipids, DNA, or proteins). Alternatively, SAM may transfer the methyl group to glycine to form sarcosine via the enzyme glycine N-methyltransferase (GNMT. After transfer of the methyl group SAM is converted to S-adenosylhomocysteine (SAH), which is broken down further to homocysteine, cystathionine and cysteine. Sarcosine may also be formed by breakdown of choline to betaine, which, after loss of a methyl group, is converted to dimethylglycine. A dehydrogenase converts dimethylglycine to sarcosine.

FIG. 4 depicts analysis of cysteine in serum with gold nanorods in accordance with various embodiments of the present invention. (A) Spectrophotometric scanning of the visible and infrared spectrum shows a distinctive red-shift in the absorbance when gold (Au) nanorods alone (dotted line) are subjected to human serum containing cysteine for 1, 4, and 6 minutes at room temperature. The arrow indicates the 950 nm wavelength at which the cysteine concentration is measured. (B) At concentration ranges of homocysteine, cystathionine, and cysteine, (analogous to that found in non-recurrent and recurrent subjects), Au nanorods were used to quantitate at 950 nm wavelength (black line). When cysteine enrichment was done prior to identical spectrophotometric detection of the same serum samples (grey line), the greater slope indicates greater sensitivity.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application. For references on how to prepare these antibodies, see D. Lane, Antibodies: A Laboratory Manual (Cold Spring Harbor Press, Cold Spring Harbor N.Y., 1988); Kohler and Milstein, (1976) Eur. J. Immunol. 6: 511; Queen et al. U.S. Pat. No. 5,585,089; and Riechmann et al., Nature 332: 323 (1988).

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For purposes of the present invention, the following terms are defined below.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy. In some embodiments, the disease condition is cancer.

“Treatment” and “treating,” as used herein refer to both therapeutic treatment and prophylactic or preventative measures, wherein the object is to prevent or slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented. Examples of cancer treatment include, but are not limited to, active surveillance, surgical intervention, prostatectomy, chemotherapy, immunotherapy, hormone therapy, radiation therapy, focal therapy, systemic therapy, high frequency ultrasound (HIFU), cryo therapy, brachytherapy, or a combination thereof.

“Tumor,” as used herein refers to all neoplastic cell growth and proliferation, whether malignant or benign, and all pre-cancerous and cancerous cells and tissues.

“Cancer” and “cancerous” refer to or describe the physiological condition in mammals that is typically characterized by unregulated cell growth. Examples of cancer include, but are not limited to B-cell lymphomas (Hodgkin's lymphomas and/or non-Hodgkins lymphomas), brain tumor, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer, and prostate cancer, including but not limited to androgen-dependent prostate cancer and androgen-independent prostate cancer.

“Chemotherapy resistance” as used herein refers to partial or complete resistance to chemotherapy drugs. For example, a subject does not respond or only partially responds to a chemotherapy drug. A person of skill in the art can determine whether a subject is exhibiting resistance to chemotherapy.

The “cystathionine synthase” comprises a polypeptide having a sequence as set forth in SEQ ID NO: 1. Also in accordance with various embodiments of the present invention, the cystathionine synthase can comprise a variant or mutant of the sequence as set forth in SEQ ID NO: 1.

The “cystathionine lyase” comprises a polypeptide having a sequence as set forth in SEQ ID NO: 2. Also in accordance with various embodiments of the present invention, the cystathionine lyase can comprise a variant or mutant of the sequence as set forth in SEQ ID NO: 2.

A “recurrence” means that the cancer has returned after initial treatment. For example, a recurrence of prostate cancer means that the prostate cancer has returned after initial treatment. When prostate cancer is caught in its earliest stages, initial therapy can lead to high chances for cure, with most men living cancer-free for five years. But prostate cancer can be slow to grow following initial therapy, and it has been estimated that about 20-30% of men will relapse after the five-year mark and begin to show signs of disease recurrence. A rising PSA is typically the first sign seen, coming well before any clinical signs or symptoms. Rise in serum PSA 0.2 ng/ml indicates biochemical recurrence. Rapidly recurrent patients were identified as those who developed biochemical recurrence following prostatectomy within 2 years (American Joint Committee on Cancer defined as having PSA≧0.2 ng/ml, confirmed at least once two weeks later). The recurrence-free population was defined as having maintained a serum PSA<0.01 ng/ml for five or more years following surgery. Being non-recurrent or recurrence-free means that the cancer is in remission; being recurrent means that the cancer is growing and/or has metastasized, and some surgery, therapeutic intervention, and/or cancer treatment is required to prevent lethality. The “non-recurrent subjects” are subjects who have non-recurrent or recurrence-free disease, and they can be used as the control for recurrent subjects who have recurrent disease or recurrence.

Prostate cancer remains the most common non-cutaneous solid malignancy in the United States, and the second leading cause of cancer specific death in men. Nevertheless, it has become increasingly clear that not all men who are diagnosed with prostate cancer require intervention. The continuing problem is that we do not know how to distinguish the estimated 80% of prostate cancer patients that may not need invasive therapy from those who need treatment at an early stage. This dilemma results in unnecessary health care cost, subjecting individuals to a major intervention (surgical or radiation) that has a clear negative impact to quality of life, and sometimes not acting soon enough for patients that need aggressive intervention.

Higher serum homocysteine, cystathionine, and cysteine concentrations independently predicted risk of early biochemical recurrence and aggressiveness of disease in a nested case control study. The methionine metabolites further supplemented known clinical variables to provide superior sensitivity and specificity in multi variable prediction models for rapid biochemical recurrence following prostatectomy. This could be especially useful for prostate cancer patients considering radiation as their primary treatment. In the current health care environment, aggressive treatments like prostatectomies and even radiation prostate ablative therapies are being reconsidered, due to cost and possible limitations in patient benefit. The biomarkers identified can potentially identify subjects who would require aggressive definitive treatment versus those who would be better served by active surveillance. Various embodiments of the present invention provide predictive biomarkers of cancer recurrence, as well as systems and methods of marker detection that can be both cost efficient and highly sensitive.

Various embodiments of the present invention provide a marker that predicts indolent disease versus recurrent and aggressive disease. Recent publications state that as many as 80% of the surgeries are unnecessary since there is a significant number of patients with indolent disease. The present invention helps us to prevent unnecessary major surgery. This is attractive as a means of saving healthcare dollars and preventing complications. For patients with recurrence disease, the current standard of care is to wait for recurrence, prior to adjuvant therapies. There is a consensus that it is too late at that point, since all adjuvant therapies are not curative. However, salvage radiation immediately following prostatectomy has been proven to prevent recurrence. Such salvage radiation is not practical for all patients, since pelvic floor radiation is associated with significant side effects and most patients may not need the aggressive therapy in the first place. Therefore, this test will help in making the decision who needs the aggressive intervention.

Various embodiments of the present invention provide unique detection methods involving cysteine detection in patient urine and serum samples using gold nanorod technology in combination of enzymes. The gold nanorod technology has not been used in the clinical setting due to the lack of specificity for thiol group containing amino acids and their metabolites, including homocysteine, cystathionine, and cysteine. Simply, the three thiol containing metabolites are of different length, to result in a broadened and diminished absorption peak due to heterogeneous assembly of the nanorods. This is more of an issue for cytathionine, since the thiol group is in a different position from that of homocysteine and cysteine. A method of converting the methionine metabolism pathway components, thereby increasing both specificity and sensitivity in serum and urine has been developed, as described herein.

According to various embodiments of the present invention, medical practitioners can use pre-surgical variables to determine if the patient is likely to have recurrent disease in order to act aggressively during surgery and immediately following surgery with adjuvant therapy, with the goal of preventing recurrence. Insurance companies and governments would appreciate that its use would save significant health care dollars in treatment and future care from complications from unnecessary surgical or radiation intervention. Urologists and Oncologists would be able to use the assay to prescribe primary and adjuvant intervention at an earlier stage in cancer progression, following definitive care, prior to any other metastasis detection method. Drug companies could use the non-invasive assay to determine if new therapies mediate a reduction in the negative prognostic factors.

Current risk stratification of patients prior to surgery involves variables including serum PSA, clinical stage, and biopsy grade. Independent serum markers in conjunction with PSA could help distinguish patients with aggressive prostate cancer. In the current era of PSA testing, clinical staging has reduced relevance when tumor volumes are relatively small. In our study, the highest biopsy Gleason score in ≧8-core biopsies provided a significant independent predictor comparable to serum cysteine and homocysteine. However, routine ultrasound directed first biopsies are reported to miss nearly a quarter of the prostate cancers [19] and often underestimate tumor grade [20,21]. The combination of serum PSA with cystathionine, cysteine, and homocysteine as markers could improve decision-making for primary treatment and earlier subsequent adjuvant therapy.

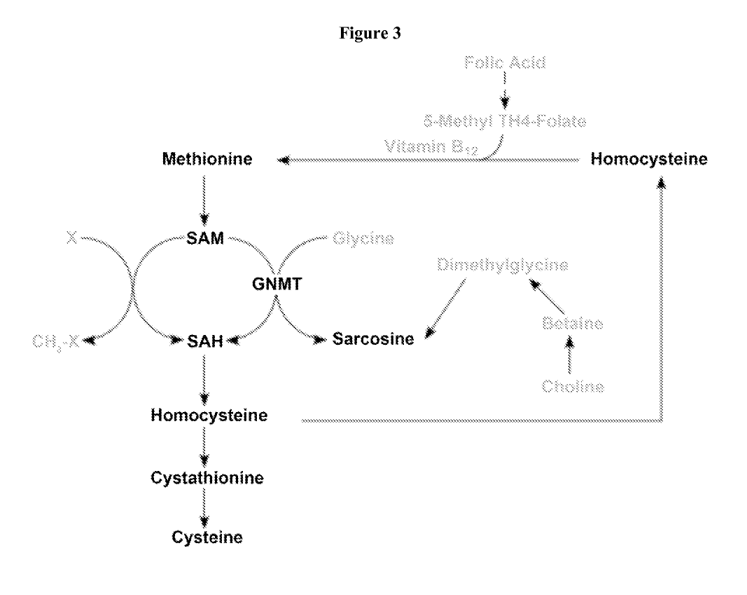

Pathways of methionine metabolism involve two mechanisms for sarcosine formation (FIG. 3). Cystathionine and cysteine are products of homocysteine catabolism important in production of glutathione. Elevation of urinary sarcosine in the absence of serum sarcosine differences was surprising, and likely the result of differential renal sarcosine excretion. Changes in sarcosine but not dimethylglycine suggest that increased activity of GNMT might have been present in the recurrent group. It is possible that for unknown reasons the recurrent group had increased S-adenosylmethionine (SAM) which activated the transulfuration pathway [22] thus, increasing cystathionine, cysteine, and formation of sarcosine. It should be noted that Sreekumar et al. [8] did not report sarcosine in patient serum or plasma associated with metastatic prostate cancer. Our data in pre-surgical subjects supports the previous report of urinary sarcosine elevation in confirmed metastatic patients. The data could mean that our patient population had previously undetected metastasis or that the elevated methionine metabolism is a precursor for metastasis. The direct role of sarcosine on metastatic progression is controversial. In contrast to the report of sarcosine directly supporting metastasis [8], a recent report suggests no association between urinary sarcosine levels and either tumor stage or Gleason score [23]. It is difficult to compare our findings with other reports since the initial study by Sreekumar et al [8] differ in the methodology of sarcosine measurement [24], sample source [25,26], and importantly criteria defining recurrence [23-27]. Our assay utilizes a stable isotope internal standard in each sample, retrieved urine and serum prior to prostate resection, and recurrence was only based on serum PSA detection. Another study compared benign controls against patients with active prostate cancer and found that urine sarcosine was only a modest predictor of disease, but when added to other new markers such as prostate cancer antigen 3 and percent-free PSA improved diagnostic power [27]. There is abundant evidence that folate and B12 deficiency and kidney disease can contribute to hyperhomocysteinemia. However, in the present investigation there was no difference in folate or methylmalonic acid levels between recurrent and non-recurrent groups. The patients in this study were accordingly recruited to minimize complicating co-morbidities. The differences we found in homocysteine, cystathionine and cysteine in serum suggest that there may be systemic metabolic differences in those patients who go on to have a biochemical relapse.

The majority of the sarcosine produced in the body is made in the liver as a downstream product of SAM and homocysteine. Studies using homozygous mice with GNMT knocked out have plasma SAM levels 50% greater than that of wild type. The SAM levels in the livers of the Gnmt null animals were 33 fold higher than in the livers of wild type animals and all of the Gnmt null animals developed hepatocellular carcinoma after 8 months [28]. Interestingly, higher cysteine values are associated with obesity [29-32]. The limited body composition data for our subject groups, however suggested little correlation of body mass index and recurrence rate. The data reported here, support increased flux through GNMT resulting in the increased formation of homocysteine and sarcosine through increased utilization of SAM. Interestingly, GNMT, is reported to be down-regulated in neoplastic tissues in general [33] including human prostate cancer [34]. Thus, the changes seen in the current investigation may not be a result of neoplastic changes in prostate tissue. While not wishing to be bound by any particular theory, we believe that these results suggest that there may be differences in the methylation capacity of different individuals or tumor hosts as a result of different levels of SAM. Unfortunately, SAM values could not be measured in the current study, because of the instability of SAM in stored serum samples. Further, it is possible that individuals with a greater methylating capacity are more likely to develop cancer leading to metastatic progression.

To our knowledge, no previous study has correlated an entire arm of a metabolic pathway in the aggressiveness of cancer. The comparison described here was made between patients with proven cancer, not between subjects with proven cancer and benign prostatic disease. These results were obtained with only a relatively small group of patients but the results are significant. The underlying biology supports the robustness of these markers. Higher serum homocysteine, cystathionine, and cysteine improved the utility of currently used clinical variables in predicting early recurrence and suggest a greater flux of methyl groups through the enzyme GNMT.

In various embodiments, the invention provides a system that comprises an isolated sample from a subject, cystathionine synthase, cystathionine lyase and nanorods. In accordance with various embodiments of the present invention, the system can be used to predict the probability of a recurrence of a cancer in a subject. In various embodiments, the subject can be human, monkey, ape, dog, cat, cow, horse, goat, pig, rabbit, mouse, or rat. In various embodiments, the sample can be serum, urine, blood, plasma, saliva, semen, lymph, or a combination thereof. In various embodiments, the cystathionine synthase comprises a polypeptide having a sequence as set forth in SEQ ID NO: 1. In various embodiments, the cystathionine lyase comprises a polypeptide having a sequence as set forth in SEQ ID NO: 2. In various embodiments, the nanorods can be made of gold, selenium, cadmium, copper, or a combination thereof.

In further embodiments, the invention provides a system that comprises an isolated sample from a subject, cystathionine synthase, cystathionine lyase, nanorods, and further comprises a PSA test, clinical stage, biopsy Gleason score, pathologic Gleason score, pathologic stage, surgical margin status, lymph node involvement, or seminal vesicle involvement, or a combination thereof. PSA level, clinical stage, and biopsy Gleason score have pre-surgical predictive value. Post-surgical standard of care information such as pathologic Gleason score, pathologic stage, surgical margin status, lymph node involvement, and seminal vesicle involvement can also augment the use of cysteine quantitation. In accordance with various embodiments of the present invention, the system can be used to predict the probability of a recurrence of a cancer in a subject. In some embodiments, the PSA test is a test of PSA velocity and/or total PSA level. PSA velocity means the rate at which PSA level rises over time.

In various embodiments, the invention provides a method of predicting the probability of a recurrence of a cancer in a subject. The method comprises: obtaining a sample from the subject; processing the sample with cystathionine synthase and cystathionine lyase; subjecting the processed sample to an assay to detect cysteine level, wherein the assay comprises nanorods; and predicting an increased probability of the recurrence of the cancer in the subject when the cysteine level in the subject is detected to be higher than in non-recurrent subjects. In various embodiments, the recurrence can be biochemical recurrence. In various embodiments, the cancer is prostate cancer, colon cancer, breast cancer, lung cancer, renal cancer, or bladder cancer. In various embodiments, the subject can be human, monkey, ape, dog, cat, cow, horse, goat, pig, rabbit, mouse, or rat. In various embodiments, the sample can be obtained before, during, or after cancer treatment. In various embodiments, the sample can be serum, urine, blood, plasma, saliva, semen, lymph, or a combination thereof. In some embodiments, the sample is urine and the urine cysteine level in the subject is above about 210 nanomoles of cysteine per milligram creatinine. In some embodiments, the sample is urine and the urine cysteine level in the subject is above about 220 nanomoles of cysteine per milligram creatinine. In some embodiments, the sample is urine and the urine cysteine level in the subject is above about 230 nanomoles of cysteine per milligram creatinine. In some embodiments, the sample is serum and the serum cysteine level in the subject is above about 400 μM of cysteine. In some embodiments, the sample is serum and the serum cysteine level in the subject is above about 410 μM of cysteine. In some embodiments, the sample is serum and the serum cysteine level in the subject is above about 420 μM of cysteine. In various embodiments, the nanorods can be made of gold, selenium, cadmium, copper, or a combination thereof.

In further embodiments, the invention provides a method of predicting the probability of a recurrence of a cancer in a subject and treating the subject. The method comprises: obtaining a sample from the subject; processing the sample with cystathionine synthase and cystathionine lyase; subjecting the processed sample to an assay to detect cysteine level, wherein the assay comprises nanorods; predicting an increased probability of the recurrence of the cancer in the subject when the cysteine level in the subject is detected to be higher than in non-recurrent subjects; and treating the subject with active surveillance, prostatectomy, chemotherapy, immunotherapy, hormone therapy, radiation therapy, focal therapy, systemic therapy, high frequency ultrasound (HIFU), cryo therapy, brachytherapy, or a combination thereof.

In various embodiments, the invention provides a method of predicting the probability of a recurrence of a cancer in a subject. The method comprises: obtaining a sample from the subject; processing the sample with cystathionine synthase and cystathionine lyase; subjecting the processed sample to an assay to detect cysteine level, wherein the assay comprises nanorods; assessing at least one additional parameter; and predicting an increased probability of the recurrence of the cancer in the subject when the cysteine level in the subject is detected to be higher than in non-recurrent subjects and/or when the additional parameter in the subject is detected to be higher or lower than in non-recurrent subjects. In various embodiments, the recurrence can be biochemical recurrence. In various embodiments, the cancer can be prostate cancer, colon cancer, breast cancer, lung cancer, renal cancer, or bladder cancer. In various embodiments, the subject can be human, monkey, ape, dog, cat, cow, horse, goat, pig, rabbit, mouse, or rat. In various embodiments, the sample can be obtained before, during, or after cancer treatment. In various embodiments, the sample can be serum, urine, blood, plasma, saliva, semen, lymph, or a combination thereof. In some embodiments, the sample is urine and the urine cysteine level in the subject is above about 210 nanomoles of cysteine per milligram creatinine. In some embodiments, the sample is urine and the urine cysteine level in the subject is above about 220 nanomoles of cysteine per milligram creatinine. In some embodiments, the sample is urine and the urine cysteine level in the subject is above about 230 nanomoles of cysteine per milligram creatinine. In some embodiments, the sample is serum and the serum cysteine level in the subject is above about 400 μM of cysteine. In some embodiments, the sample is serum and the serum cysteine level in the subject is above about 410 μM of cysteine. In some embodiments, the sample is serum and the serum cysteine level in the subject is above about 420 μM of cysteine. In various embodiments, the nanorods can be made of gold, selenium, cadmium, copper, or a combination thereof.

In further embodiments, the invention provides a method of predicting the probability of a recurrence of a cancer in a subject and treating the subject. The method comprises: obtaining a sample from the subject; processing the sample with cystathionine synthase and cystathionine lyase; subjecting the processed sample to an assay to detect cysteine level, wherein the assay comprises nanorods; assessing at least one additional parameter; predicting an increased probability of the recurrence of the cancer in the subject when the cysteine level in the subject is detected to be higher than in non-recurrent subjects and/or when the additional parameter in the subject is detected to be higher or lower than in non-recurrent subjects; and treating the subject with active surveillance, prostatectomy, chemotherapy, immunotherapy, hormone therapy, radiation therapy, focal therapy, systemic therapy, high frequency ultrasound (HIFU), cryo therapy, brachytherapy, or a combination thereof.

In further embodiments, the invention provides a method of predicting the probability of a recurrence of a cancer in a subject. The method comprises: obtaining a sample from the subject; processing the sample with cystathionine synthase and cystathionine lyase; subjecting the processed sample to an assay to detect cysteine level, wherein the assay comprises nanorods; assessing at least one additional parameter; and predicting an increased probability of the recurrence of the cancer in the subject when the cysteine level in the subject is detected to be higher than in non-recurrent subjects and/or when the additional parameter in the subject is detected to be higher or lower than in non-recurrent subjects. In some embodiments, the additional parameter is PSA velocity, PSA level, pre-surgical PSA level, post-surgical PSA level, pre-treatment PSA level, post-treatment PSA level, biopsy Gleason score, clinical stage, number of positive cores, number of negative cores, Karnofsky performance status, Hemoglobin value, Lactate dehydrogenase value, Alkaline phosphatase value, Albumin level, urinary albumin level, or urinary creatinine level, or a combination thereof. Urinary albumin level and urinary creatinine level can also be used to assess if the subject has good liver and kidney functions. Urinary creatinine level can also be used to normalize differences in urine volume when measuring urinary cysteine levels. In some further embodiments, the additional parameter is a pre-treatment parameter comprising pre-treatment PSA level, pre-treatment biopsy Gleason Score, pre-treatment clinical stage, pre-treatment urinary albumin level, or pre-treatment urinary creatinine level, or a combination thereof. In some embodiments, the PSA level in the subject is above about 6.0 ng/ml in serum. In some embodiments, the Gleason score in the subject is above 7.

In various embodiments, the invention provides a method of predicting the probability of a recurrence of a cancer in a subject. The method comprises: obtaining a sample from the subject; processing the sample with cystathionine synthase and cystathionine lyase; subjecting the processed sample to an assay to detect cysteine level; and predicting an increased probability of the recurrence of the cancer in the subject when the cysteine level in the subject is detected to be higher than in non-recurrent subjects. In various embodiments, the recurrent can be biochemical recurrence. In various embodiments, the cancer can be prostate cancer, colon cancer, breast cancer, lung cancer, renal cancer, or bladder cancer. In various embodiments, the subject can be human, monkey, ape, dog, cat, cow, horse, goat, pig, rabbit, mouse, or rat. The sample can be obtained before, during, or after cancer treatment. In various embodiments, the sample can be serum, urine, blood, plasma, saliva, semen, lymph, or a combination thereof. In some embodiments, the sample is urine and the urine cysteine level in the subject is above about 210 nanomoles of cysteine per milligram creatinine. In some embodiments, the sample is urine and the urine cysteine level in the subject is above about 220 nanomoles of cysteine per milligram creatinine. In some embodiments, the sample is urine and the urine cysteine level in the subject is above about 230 nanomoles of cysteine per milligram creatinine. In some embodiments, the sample is serum and the serum cysteine level in the subject is above about 400 μM of cysteine. In some embodiments, the sample is serum and the serum cysteine level in the subject is above about 410 μM of cysteine. In some embodiments, the sample is serum and the serum cysteine level in the subject is above about 420 μM of cysteine.

In further embodiments, the invention provides a method of predicting the probability of a recurrence of a cancer in a subject and treating the subject. The method comprises: obtaining a sample from the subject; processing the sample with cystathionine synthase and cystathionine lyase; subjecting the processed sample to an assay to detect cysteine level; predicting an increased probability of the recurrence of the cancer in the subject when the cysteine level in the subject is detected to be higher than in non-recurrent subjects; and treating the subject with active surveillance, prostatectomy, chemotherapy, immunotherapy, hormone therapy, radiation therapy, focal therapy, systemic therapy, high frequency ultrasound (HIFU), cryo therapy, brachytherapy, or a combination thereof.

In various embodiments, the invention provides a system that comprises cystathionine synthase, cystathionine lyase and nanorods. In accordance with various embodiments of the present invention, the system can be used to detect a cysteine level in a sample from a subject. In various embodiments, the subject can be human, monkey, ape, dog, cat, cow, horse, goat, pig, rabbit, mouse or rat. In various embodiments, the sample can be serum, urine, blood, plasma, saliva, semen, lymph, or a combination thereof. In various embodiments, the cystathionine synthase comprises a polypeptide having a sequence as set forth in SEQ ID NO: 1. In various embodiments, the cystathionine lyase comprises a polypeptide having a sequence as set forth in SEQ ID NO: 2. In various embodiments, the nanorods can be made of gold, selenium, cadmium, copper, or a combination thereof.

In further embodiments, the invention provides a system that comprises cystathionine synthase, cystathionine lyase, nanorods, and further comprise a PSA test, clinical stage, biopsy Gleason score, pathologic Gleason score, pathologic stage, surgical margin status, lymph node involvement, or seminal vesicle involvement, or a combination thereof. PSA level, clinical stage, and biopsy Gleason score have pre-surgical predictive value. Post-surgical standard of care information such as pathologic Gleason score, pathologic stage, surgical margin status, lymph node involvement, and seminal vesicle involvement can also augment the use of cysteine quantitation. In accordance with various embodiments of the invention, the system can be used to predict the probability of a recurrence of a cancer in a subject. In some embodiments, the PSA test is a test of PSA velocity and/or total PSA level. PSA velocity means the rate at which PSA level rises over time.

In various embodiments, the invention provides a method of detecting a cysteine level in a sample from a subject. The method comprises: obtaining a sample from the subject; processing the sample with cystathionine synthase and cystathionine lyase; mixing the processed sample with nanorods which forms a reaction mixture; measuring a change of absorption spectrum of the reaction mixture; and detecting the cysteine level based upon the change of absorption spectrum. In various embodiments, the sample can be serum, urine, blood, plasma, saliva, semen, lymph, or a combination thereof. In various embodiments, the subject can be human, monkey, ape, dog, cat, cow, horse, goat, pig, rabbit, mouse or rat. In various embodiments, the cystathionine synthase comprises a polypeptide having a sequence as set forth in SEQ ID NO: 1. In various embodiments, the cystathionine lyase comprises a polypeptide having a sequence as set forth in SEQ ID NO: 2. In various embodiments, the nanorods can be made of gold, selenium, cadmium, copper, or a combination thereof.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

We evaluated the serum and urine of radical prostatectomy patients for metabolites to differentiate those who developed early biochemical recurrence (rise in serum PSA 0.2 ng/ml) within two years of surgery and those who remained recurrence-free after more than five years. We found that the urine of patients in the rapidly recurrent group had significantly higher concentrations of sarcosine and cysteine than those in the recurrence-free group. In addition, significantly greater concentrations of serum cystathionine, homocysteine and cysteine were found in the rapidly recurrence group compared to the recurrence-free group. These products of elevated methionine catabolism in patients with rapidly recurrent prostate cancer represent pre-surgical indicators that augmented serum PSA for the prediction of clinically significant prostate cancer.

We have developed a serum test with high sensitivity and specificity for patients diagnosed with prostate cancer that predicts recurrent cancer prior to surgical intervention. This finding has implications for patients with the highest chance of developing metastatic progression. If a urologist or oncologist knows a patient is more or less likely to have aggressive cancer the mode of intervention can be personalized. Prostate cancer patients uniquely benefit from such information since majority of patients harbor an indolent localized disease. Active surveillance can then be an informed option patients can make. Similarly, prostate cancer patients are normally not given adjuvant therapy until after frank recurrence detection. At this stage, all treatment options are non-curative. Early aggressive therapy is reported by multiple groups to have significant survival benefit for high risk patients.

### Example 1

Urine and serum samples (n=54 and 58, respectively), collected at the time of prostatectomy were divided into subjects who developed biochemical recurrence within 2 years and those who remained recurrence-free after 5 years. Multiple methionine metabolites were measured in urine and serum by GC-MS. The role of serum metabolites and clinical variables (biopsy Gleason grade, clinical stage, serum prostate specific antigen [PSA]) on biochemical recurrence prediction were evaluated. Urinary sarcosine and cysteine levels were significantly higher (p=0.03 and p=0.007 respectively) in the recurrent group. However, in serum, concentrations of homocysteine (p=0.003), cystathionine (p=0.007) and cysteine (p<0.001) were more abundant in the recurrent population. The inclusion of serum cysteine to a model with PSA and biopsy Gleason grade improved prediction over the clinical variables alone (p<0.001).

Higher serum homocysteine, cystathionine, and cysteine concentrations independently predicted risk of early biochemical recurrence and aggressiveness of disease in a nested case control study. The methionine metabolites further supplemented known clinical variables to provide superior sensitivity and specificity in multivariable prediction models for rapid biochemical recurrence following prostatectomy.

### Example 2

### A. Ethics Statement.

This nested case-control study was conducted in accordance with the Institutional Review Board of Vanderbilt University. Written consent was given by the patients for their information to be stored in the hospital database. The board specifically approved the research use of the di-identified information and “on the shelf” specimens to be used for research under a waiver of consent.

### B. Patient Selection

The digital medical records of 400 subjects were retrospectively examined using the Vanderbilt University Department of Urologic Surgery registry of radical prostatectomies performed between 2003 and 2007. Several patients were excluded for reasons of compromised renal, heart, or liver function as was determined by electronic records of elevated urinary creatinine, hypertension, cardiac infarction history, and blood markers for hepatic function. Additionally, availability of follow-up data and records of pre-surgical hormone-ablation therapy were reasons for exclusion. Rapidly recurrent patients were identified as those who developed biochemical recurrence following prostatectomy within 2 years (American Joint Committee on Cancer defined as having PSA≧0.2 ng/ml, confirmed at least once two weeks later). The recurrence-free population was defined as having maintained a serum PSA<0.01 ng/ml for five or more years following surgery. Ultimately, for this nested case control study we focused on 54 subjects for analysis of urine and 58 subjects for analysis of serum who developed rapid biochemical recurrence and an age-matched recurrence-free control group who were free of recurrence. The mean age for the subjects was 60 years. All subjects were annotated based on age, pre-surgical serum PSA, biopsy Gleason score, clinical stage, and detection of biochemical recurrence.

### C. Urine and Serum Quantitative Metabolic Analysis.

Serum and urine obtained at the time of radical prostatectomy were rapidly processed and stored at −80° C. We evaluated serum and urine for the metabolites, sarcosine, dimethylglycine, methionine, homocysteine, cystathionine, cysteine, methylmalonic acid and methylcitrate by gas-liquid chromatography/mass spectrometry [11,12,13]. Folate was measured microbiologically as described by Horne [14]. Urinary metabolites were expressed as nmol/mg creatinine to correct for differences in urine volume. Creatinine in urine was measured by the Jaffe method [15].

### D. Statistical Analysis.

Patients' baseline demographic and clinical variables were assessed using Wilcoxon rank sum tests for continuous variables and Fisher exact tests for categorical (including binary) variables. All marker values, as well as PSA levels, were logarithmically transformed to achieve normality. Correlations among the markers were assessed using Spearman's rank correlation. Logistic regression models were used to analyze incidence of recurrence. The base model includes serum PSA, biopsy Gleason score, and clinical stage, clinical variables that are available prior to surgery. The post-surgical variables (e.g., lymph nodes, surgical margins, pathologic Gleason scores) were not considered. For multiplicity control, p≦0.007 (p-value less than 5%/7=0.7%) was considered statistically significant. To avoid further overfitting of the data, no variable selection was performed in the subsequent analyses based on logistic regression models. We used a likelihood ratio test to compare the simpler model (without the metabolites) and the full model (with the individual metabolites). Receiver operating characteristics (ROC) curves were generated for each logistic regression model, where the area under ROC curve (AUC) was determined. Integrated discrimination improvement (IDI) and Net reclassification index (NRI) [16] were used to compare the models' ability to distinguish recurrence and non-recurrence. The logrank tests were used to assess the difference in recurrence-free survival between the two groups illustrated by Kaplan-Meier plots. For the selected markers, Cox proportional hazard regression models were fit, and likelihood ratio tests were used to assess markers' association with time to recurrence outcome. The proportional hazard assumption was assessed using the method of Grambsch and Therneau [17]. All data analyses were performed using R 2.10.1 (R Development Core Team, Vienna, Austria); a significance level of 0.05 was used for statistical inference unless otherwise noted.

### E. Enzymes and Protein Sequences

An example of protein sequence of the cystathionine synthase is SEQ ID NO:1 (Protein: Cystathionine beta-synthase 305 amino acids; Source organism: Helicobacter pylori 908; ACCESSION: ADN79248).

MILTAMQDAIGRTPIFKFTRKDYPIPLKSAIYAKLEHLNPGGSVKDRLGQYLIKEA
FRTHKITSTTTIIEPTAGNTGIALALVAIKHHLKTIFVVPEKFSVEKQQIMRALGALVINTP
TSEGISGAIKKSKELAESIPDSYLPLQFENPDNPAAYYHTLAPEIVKELGTNFTSFVAGIGS
GGTFAGTAKYLKERIPNIRLIGVEPEGSILNGGEPGPHEIEGIGVEFIPPFFANLDIDGFETIS
DEEGFSYTRKLAKKNGLLVGSSSGAAFAAALKEVQRLPEGSQVLTIFPDMADRYLSKGI
YS

An example of protein sequence of the cystathionine lyase is SEQ ID NO:2 (Protein: Cystathionine gamma-lyase 378 amino acids; Source organism: Helicobacter pylori 908; ACCESSION: ADN79247).

MQTKLIHGGISEDATTGAVSVPIYQASTYRQDAIGRHKGYEYSRSGNPTRFALEE
LIADLEGGVKGFAFASGLAGIHAVFSLLQSGDHVLLGDDVYGGTFRLFNKVLVKNGLSC
TIIDTSDISQIKKAIKPNTKALYLETPSNPLLKITDLAQCASVAKDHGLLTIVDNTFATPYC
QNPLLLGADIVAHSGTKYLGGHSDVVAGLVTTNNEALAQEIAFFQNAIGGVLGPQDSW
LLQRGIKTLGLRMEAHQKNALCVAEFLEKHPKVERVYYPGLPTHPNHELAKAQMRGFS
GMLSFTLKNDSEAALFVESLKLFILGESLGGVESLVGIPALMTHACIPKEQREAAGIRDGL
VRLSVGIEHEQDLLEDLEQAFAKIS

### F. Cysteine Detection

Urine: (1) 1 ml of urine is needed for the analysis. Creatine and albumin levels is measured using 200 μl for each assay. Elevated creatine and albumin (>1.2 mg/dL and >8 mg/dL, respectively) would exclude the use of the cysteine assay for the subject. (2) Of the remaining 600 μA, 200 μl in triplicate is used for cysteine measurement. To each of the tubes, the following will be added: serine, pyridoxal phosphate, cystathionine beta-synthase, and cystathione gamma-lyase. This reaction is allowed to proceed for 20 min at room temperature. (3) Since the recombinant enzymes (of Helicobacter pylori) have a glutathione S transferase (GST) tag modification, glutathione bound sepharose beads (10 μl) is added to the reaction. Following 5 min. incubation on ice, the tubes are centrifuged briefly. The supernatant (free of the modifying enzymes) is transferred to wells of a 96 well plate. (4) Gold nanorods [10 pM, can replaced with SeCd rods having Au ends] with an aspect ratio of 10 nm×30 nm are added to the analyte and allowed to react for 10 min at room temperature. Importantly, if gold is used instead of SeCd rods, the rods need to be protected with cetyltrimethylammonium bromide (CTAB) prior to analysis. Following the 10 min incubation with the nanorods, HCl [0.2 mM] is added. The absorption spectra is recorded on a 96 well plate reader with dynamically from 2 min to 8 min. following HCl addition at 950 nm wavelength. Similar readings can be had by 1 cm path length cuvette if samples are analyzed individually.

Serum: for detection of cysteine in serum, each of the steps above is the same. As above, urinary creatine and albumin levels is need to determine eligibility for the test.

### Example 3

Methionine metabolites support prediction of biochemical recurrence—Urine metabolites were initially measured in fifty-four patients who developed biochemical recurrence (N=25) and those that remained recurrence-free (N=29). These patients were matched for age and pre-surgical serum PSA. Table 1 enumerates the clinical characteristics of the two patient groups by serum PSA, clinical stage, and biopsy Gleason grade. Majority of patients had a clinical stage of T1. Creatinine-normalized urinary dimethylglycine and homocysteine were not significantly different between the two groups. However, we found urinary sarcosine to be significantly elevated at the time of surgery in patients who developed biochemical recurrence, as originally reported for patients with frank prostate metastatic lesions [8]. We further found that urinary cysteine was significantly elevated in biochemically-recurrent patients compared to those who remained recurrence-free five years following prostatectomy. Urine analysis in a pre-surgical patient population suggested products of methionine catabolism might correlate with prostate cancer progression status.

TABLE 1
Table 1: The values for methionine metabolites measured in the
urine of the recurrent-free and the recurrent groups are compared.
Values for sarcosine, homocysteine, dimethylglycine and cysteine
are expressed as μmoles/mg creatinine. Wilcoxon rank sum tests
for continuous variables and Fisher exact tests for categorical
(including binary) variables are indicated. Normal values for
metabolites (nmole/mg creatinine) are: cysteine, 140-579; homocysteine,
0.974-7.17; dimethylglycine, 10.1-108.2 and sarcosine, 2.65-8.67.
Median values with quartiles were used to summarize the distributions
of the continuous variables.

| | Recurrent-free | | Recurrent | | P |
|---|---|---|---|---|---|
| | (29) | | (25) | | value |
| Age | 59 | (53, 64) | 62 | (58, 67) | 0.10 |
| Pre-surgery PSA | 5.2 | (4.3, 6.5) | 6.0 | (5.0, 8.2) | 0.08 |
| Clinical stage | | | | | 0.09 |
| (N = 16/18) | | | | | |
| T1 | 15 | (94%) | 12 | (67%) | |
| T2 | 1 | (6%) | 6 | (33%) | |
| T3 | 0 | | 0 | | |
| Biopsy Gleason | | | | | 0.050 |
| (N = 16/18) | | | | | |
| 4 | 1 | (6%) | 0 | | |
| 5 | 2 | (12%) | 0 | | |
| 6 | 9 | (56%) | 4 | (22%) | |
| 7 | 3 | (19%) | 8 | (44%) | |
| 8 | 1 | (6%) | 3 | (17%) | |
| 9 | 0 | | 2 | (11%) | |
| 10 | 0 | | 1 | (6%) | |
| Urine cysteine | 190 | (168, 212) | 221 | (189, 252) | 0.007 |
| (N = 29/24) | | | | | |
| Urine homocysteine | 2.7 | (2.2, 3.2) | 2.8 | (2.4, 4.0) | 0.40 |
| Urine dimethylglycine | 27.3 | (22.1, 38.5) | 25.4 | (17.6, 33.7) | 0.34 |
| Urine sarcosine | 3.7 | (3.1, 5.7) | 5.4 | (4.1, 6.7) | 0.03 |

We then performed a nested case control study with pre-surgical serum. Fifty-eight age-matched prostatectomy patients were stratified by pre-surgical PSA, clinical stage, and biopsy Gleason grade as well as pathologic variables (Table 2). As expected, clinical variables were significantly different in the two populations, as were the post-surgical pathologic factors. Interestingly, the serum homocysteine, cystathionine, and cysteine were significantly higher in the biochemically-recurrent patients (p value<0.001). However, clinical stage and serum levels of sarcosine, dimethylglycine, folate, methylcitrate, and methylmalonic acid were not significantly different between the two populations. Normal methylcitrate levels in both populations supported renal sufficiency. Serum methylmalonic acid levels, an indicator of vitamin B-12 status [18], were not different between the two groups. Serum and urine cysteine correlation did not reach statistical significance (p=0.06, Table 3). However, serum homocysteine was strongly correlated with cysteine (Spearman's rank correlation=0.65, p<0.01). Therefore, the higher serum homocysteine was not a function of differences in renal function, vitamin B-12 or folate status.

TABLE 2
Table 2: The values for methionine metabolites measured in the
sera of the recurrent-free and the recurrent groups are compared.
Wilcoxon rank sum tests for continuous variables and Fisher exact
tests for categorical (including binary) variables are indicated.
Normal values for metabolites are: cysteine, 203-369 μM homocysteine,
5.4-13.9 μM; dimethylglycine, 1.4-5.3 μM; sarcosine, 0.6-
2.7 μM; methionine, 11.3-42.7 μM; folate, >3.0 ng/ml;
methylcitrate, 60-228 nM; methylmalonate, 73-271 nM; cystathionine,
44-342 nM. Median values with quartiles were used to summarize
the distributions of the continuous variables.

| | Recurrent-free | | Recurrent | | P |
|---|---|---|---|---|---|
| | (30) | | (28) | | value |
| Age | 59 | (54, 64) | 61 | (59, 64) | 0.07 |
| Pre-surgery PSA | 5.4 | (4.0, 8.1) | 6.8 | (5.2, 8.9) | 0.02 |
| Clinical stage | | | | | 0.30 |
| T1 | 24 | (80%) | 18 | (64%) | |
| T2 | 6 | (20%) | 9 | (32%) | |
| T3 | 0 | | 1 | (4%) | |
| Biopsy Gleason | | | | | 0.006 |
| 4 | 1 | (3%) | 0 | | |
| 5 | 2 | (7%) | 0 | | |
| 6 | 18 | (60%) | 6 | (20%) | |
| 7 | 6 | (20%) | 13 | (46%) | |
| 8 | 2 | (7%) | 4 | (15%) | |
| 9 | 1 | (3%) | 4 | (15%) | |
| 10 | 0 | | 1 | (4%) | |
| Serum cysteine | 346 | (321, 377) | 419 | (367, 452) | <0.001 |
| Serum | 9.0 | (8.0, 10.2) | 11.7 | (9.4, 13.4) | 0.003 |
| homocysteine | | | | | |
| Serum | 4.6 | (3.8, 4.7) | 4.9 | (4.2, 5.4) | 0.21 |
| dimethylglycine | | | | | |
| (n = 27/23) | | | | | |
| Serum sarcosine | 1.3 | (1.1, 1.4) | 1.3 | (1.1, 1.7) | 0.67 |
| (n = 27/23) | | | | | |
| Serum methionine | 24.8 | (21.7, 30.6) | 27.6 | (23.9, 33.7) | 0.08 |
| (n = 27/27) | | | | | |
| Serum folate | 44.8 | (25.2, 52.8) | 42.3 | (31.3, 51.5) | 0.72 |
| (n = 27/28) | | | | | |
| Serum | 126 | (102, 144) | 135 | (117, 167) | 0.13 |
| methylcitrate | | | | | |
| Serum | 167 | (145, 220) | 164 | (146, 211) | 0.91 |
| methylmalonate | | | | | |
| Serum | 149 | (130, 176) | 186 | (148, 239) | 0.007 |
| cystathionine | | | | | |
| (n = 29/26) | | | | | |
| Lymph node | 0 | (0%) | 6 | (21%) | 0.01 |
| involvement | | | | | |
| SV involvement | 0 | (0%) | 8 | (29%) | 0.002 |
| Positive | 1 | (3%) | 8 | (29%) | 0.01 |
| surgical margin | | | | | |
| Stage III+ | 3 | (10%) | 21 | (75%) | <0.001 |
| Pathologic | | | | | 0.002 |
| Gleason | | | | | |
| 5 | 2 | (7%) | 0 | (0%) | |
| 6 | 15 | (50%) | 4 | (14%) | |
| 7 | 10 | (33%) | 14 | (50%) | |
| 8 | 3 | (10%) | 4 | (14%) | |
| 9 | 0 | (0%) | 6 | (21%) | |

TABLE 3
Table 3. Correlations between serum and urine markers. All
correlations are rank based “Spearman's rho”.

| | Correlation coefficient | P value | n |
|---|---|---|---|
| Sarcosine | 0.19 | 0.34 | 28 |
| Dimethylglycine | 0.12 | 0.53 | 28 |
| Cysteine | 0.33 | 0.06 | 33 |
| Homocysteine | 0.13 | 0.48 | 34 |

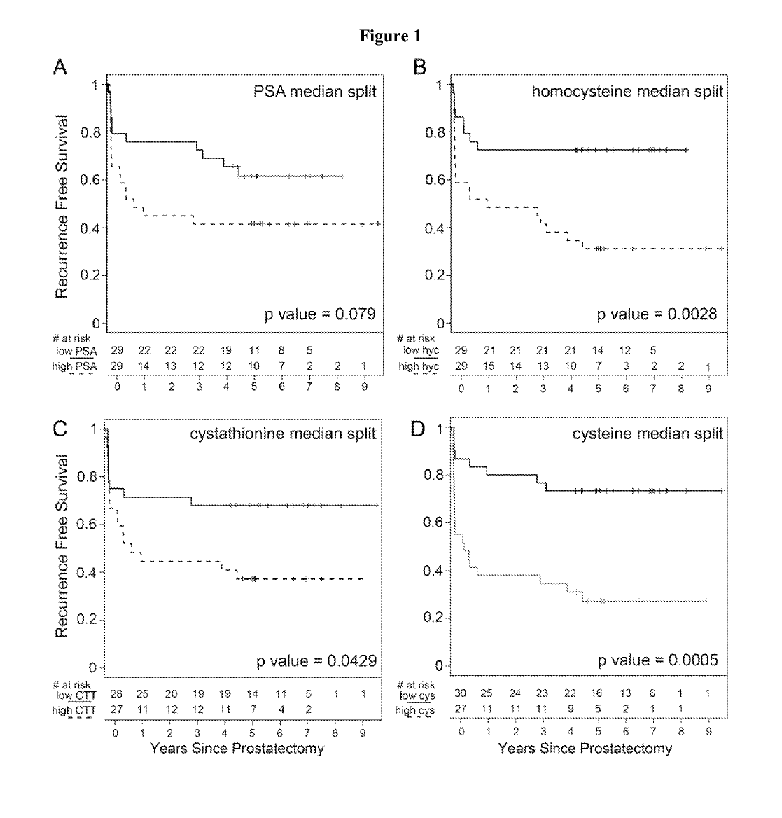

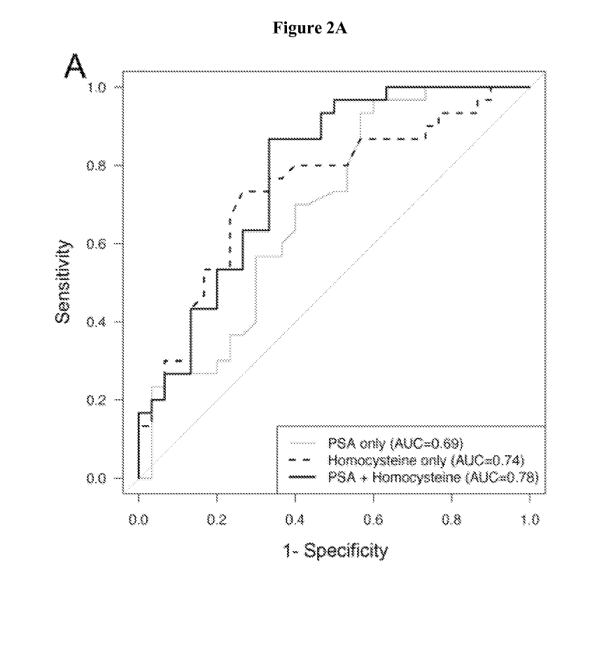

The relevance of these newly identified markers to patient recurrence status were illustrated in Kaplan-Meier plots for homocysteine, cystathionine, and cysteine as compared to pre-operative serum PSA levels, and time-to-recurrence (FIG. 1). Each of the markers could separate rapidly recurrent from the recurrence-free progression. However, serum cysteine detection had the greatest discriminatory power in the two populations prior to prostatectomy.

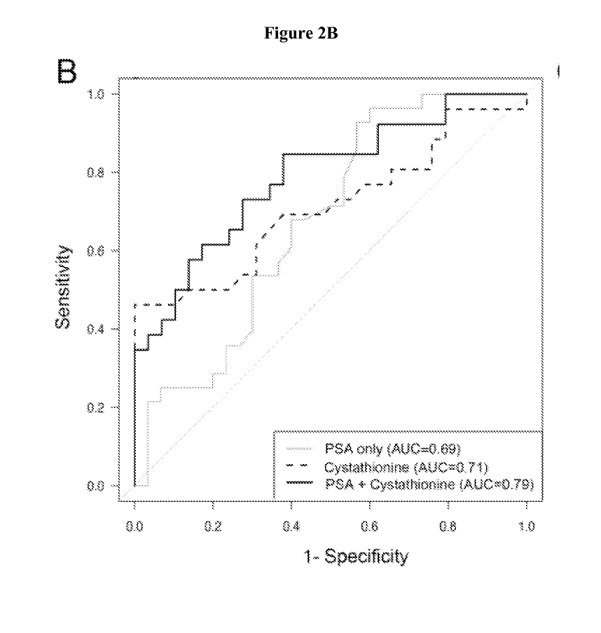

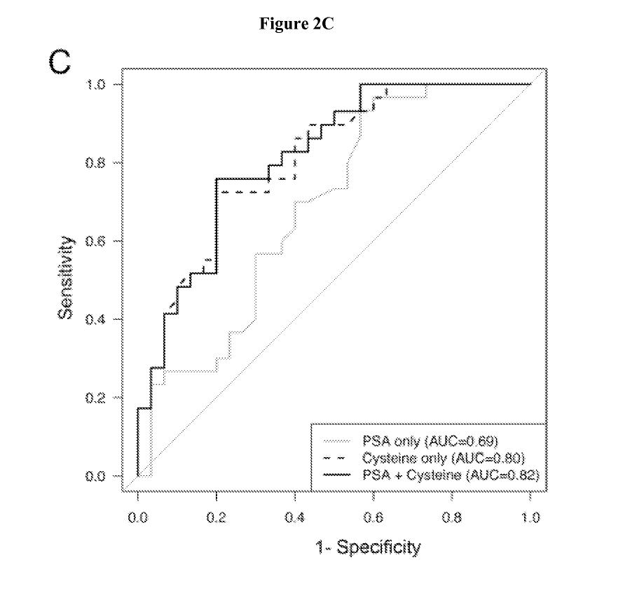

The clinical value of these methionine metabolites as biomarkers would be to significantly increase the ability to predict aggressive prostate cancer features and early biochemical recurrence over and above existent clinical variables including serum PSA, biopsy Gleason score, and clinical stage. We developed a multiple logistic regression model for the prediction of biochemical recurrence based on serum methionine metabolites and the pre-surgical predictor variables, serum PSA and biopsy Gleason grade. Since majority of patients in both cohorts had clinical stage Tlc disease, this variable had little discriminative power and was dropped from the model. Serum cysteine, cystathionine, and homocysteine were the top three predictors for recurrence in 70% of the patients, so further analysis of methionine metabolites focused on these three metabolites. Correlations between cysteine and homocysteine were the highest among all pair-wise correlations (R2=0.65, p<0.01), and cysteine was also highly correlated with cystathionine (R2=0.39, p<0.01, Table 4). Addition of serum homocysteine provided the greatest improvement of the logistic regression models compared to the base model with PSA and biopsy Gleason (p=0.0007), followed by cysteine (p=0.0017), and cystathionine (p=0.0037). Correlation between cystathionine and homocysteine was moderate (R2=0.22, p=0.10). Based on multiple logistic regression models (Table 5), odds of recurrence increased 5.79 fold (95% CI: 1.65 to 20.29, p=0.006) when cysteine levels increased from 343 (lower quartile, henceforth Q1) to 436 (upper quartile, henceforth Q3). This logistic regression model did not find pre-surgical serum PSA levels to be significantly associated with recurrence status. In a separate model, cystathionine levels were significantly associated with recurrence status. Odds of recurrence were 2.44 (95% CI: 1.07 to 5.56, p=0.03) times higher when cystathionine levels were increased from 139 (Q1) to 200 (Q3). Serum PSA levels were marginally associated with recurrence in this model; the odds ratio was 2.94 (95% CI: 1.02 to 8.48, p=0.046) when PSA levels were increased from 4.7 (Q1) to 8.5 (Q3). Homocysteine levels were also found to be associated with recurrence status. In all of these models biopsy Gleason grade was significantly associated with recurrence. To evaluate the additional utility of these three markers, the models including cysteine, cystathionine, or homocysteine in addition to serum PSA levels and biopsy Gleason grade were compared to a model utilizing PSA plus biopsy Gleason only. Clinical stage values did not contribute to the improvement of the models. Area under the ROC curves were similar (AUC=0.86) for the cysteine, cystathionine, and homocysteine when combined with the clinical variables and significantly superior to the clinical variables alone (AUC=0.81). The Integrated Discrimination Improvement (IDI) and Net Reclassification Improvement (NRI) supported the statistical significance of the improvement (Table 6). The benefit of these metabolites as combined with the standard PSA test is evident when PSA sensitivity and specificity were compared to a combined prediction of biochemical recurrence by the ROC in FIG. 2 following prostatectomy, using only serum PSA. The AUC with only serum markers were similar to the more comprehensive ones including biopsy results. There was a significant association between these markers and recurrence status; however the markers did not necessarily indicate usefulness in predicting recurrence-free survival.

TABLE 4
Table 4. Correlations among serum markers. All
correlations are rank based “Spearman's rho”, presented
as correlation, p-value, and n.

| | Dimethyl- | | | Cysta- |
|---|---|---|---|---|
| | glycine | Sarcosine | Cysteine | thionine |
| Homocysteine | 0.28, 0.05 | 0.28, 0.04 | 0.65, <0.01 | 0.22, 0.10 |
| | n = 50 | n = 50 | n = 57 | n = 55 |
| Dimethylglycine | | 0.35, 0.01 | 0.40, <0.01 | 0.16, 0.26 |
| | | n = 50 | n = 50 | n = 48 |
| Sarcosine | | | 0.35, <0.01 | 0.08, 0.60 |
| | | | n = 50 | n = 48 |
| Cysteine | | | | 0.39, <0.01 |
| | | | | n = 54 |

TABLE 5
Table 5: Logistic regression models.

| | | | | |
|---|---|---|---|---|
| | Comparison | | 95% | P |
| Variable | Q3:Q1 | Odds | Confidence Int. | value |

SERUM HOMOCYSTEINE MODEL

| | | | | |
|---|---|---|---|---|
| Pre-surgery PSA | 8.5:4.7 | 2.39 | (0.90, 6.33) | 0.080 |
| Biopsy GS | 7:6 | 4.29 | (1.59, 11.56) | 0.004 |
| Serum homocysteine | 12.5:8.6 | 4.74 | (1.61, 13.90) | 0.005 |

SERUM CYSTATHIONINE MODEL

| | | | | |
|---|---|---|---|---|
| Pre-surgery PSA | 8.5:4.7 | 2.94 | (1.02, 8.48) | 0.046 |
| Biopsy GS | 7:6 | 2.80 | (1.24, 6.28) | 0.013 |
| Serum cystathionine | 200:139 | 2.44 | (1.07, 5.56) | 0.033 |

SERUM CYSTEINE MODEL

| | | | | |
|---|---|---|---|---|
| Pre-surgery PSA | 8.5:4.7 | 1.82 | (0.66, 4.96) | 0.245 |
| Biopsy GS | 7:6 | 2.51 | (1.19, 5.31) | 0.015 |
| Serum cysteine | 436:343 | 5.79 | (1.65, 20.29) | 0.006 |

TABLE 6
Table 6: The Integrated Discrimination Improvement (IDI) and
Net Reclassification Improvement (NRI) were summarized below,
supporting the statistical significance of the improvement.

| | | | P- | | | P- |
|---|---|---|---|---|---|---|
| | IDI | 95% CI | value | NRI | 95% CI | value |
| Homocysteine | 0.14 | 0.05-0.24 | 0.003 | 1.03 | 0.52-1.55 | <0.001 |
| Cystathionine | 0.12 | 0.004-0.20 | 0.003 | 0.81 | 0.28-1.34 | 0.003 |
| Cysteine | 0.14 | 0.04-0.23 | 0.005 | 0.64 | 0.13-1.16 | 0.015 |

To define the efficacy of the markers in predicting recurrence-free survival, Cox proportional hazard regression models were fit showing that cysteine, cystathionine, and homocysteine were each independent predictors of recurrence-free survival when adjusting for pre-operative serum PSA and biopsy Gleason score (Table 7). Specifically, serum cysteine, cystathionine, and homocysteine values increased (p<0.001, p=0.014, p<0.001, respectively) with increased risk of recurrence on multivariable analysis with adjustment for both serum PSA and biopsy Gleason score.

TABLE 7
Table 7: Cox regression models

| | | | | |
|---|---|---|---|---|
| | Comparison | | 95% | P |
| Variable | Q3:Q1 | Hazard | Confidence Int. | value |

SERUM HOMOCYSTEINE MODEL

| | | | | |
|---|---|---|---|---|
| Pre-surgery PSA | 8.5:4.7 | 2.34 | (1.27, 4.32) | 0.007 |
| Biopsy GS | 7:6 | 2.01 | (1.44, 2.79) | <0.001 |
| Serum homocysteine | 12.5:8.6 | 2.43 | (1.48, 4.01) | <0.001 |

SERUM CYSTATHIONINE MODEL

| | | | | |
|---|---|---|---|---|
| Pre-surgery PSA | 8.5:4.7 | 2.47 | (1.30, 4.70) | 0.006 |
| Biopsy GS | 7:6 | 1.64 | (1.21, 2.22) | 0.001 |
| Serum cystathionine | 200:139 | 1.69 | (1.11, 2.57) | 0.014 |

SERUM CYSTEINE MODEL

| | | | | |
|---|---|---|---|---|
| Pre-surgery PSA | 8.5:4.7 | 2.00 | (1.03, 3.86) | 0.039 |
| Biopsy GS | 7:6 | 1.71 | (1.24, 2.37) | 0.001 |
| Serum cysteine | 436:343 | 2.59 | (1.51, 4.43) | <0.001 |

### Example 4

As shown in FIG. 3, cysteine is the last step of the methionine metabolism pathway. Cysteine is the most abundant in both urine and serum and is the most reflective of alterations in any component of the pathway that patients have. Cysteine is a superior serum or urine-based predictor of biochemical recurrence following prostatectomy than any previous report.

The current standard for cysteine detection involves gas chromatography mass spectrometry. It involves the use of radio-labeled metabolites for the development of a standard curve and subsequent detection of the metabolites in the patient samples. This process is a highly complex, labor intensive and costly ($800 per patient sample). Here we developed a simple and low-cost detection method of cysteine level for predicting the probability of cancer recurrence.

Gold nanorods have not been used for detection of cysteine in serum in a clinical setting. The technology is solely based on the fact that thiol groups (—SH) found in cysteine bind to the gold and cause the nanorods to align linearly to result in a change in light absorption detected by a spectrophotometer. However, in a clinical setting where cysteine is not the only thiol containing molecule in the serum or urine, the nanorods cannot distinguish one from another. For example if homocysteine (also having a free thiol group available for gold rod interaction) is in the sample it could interfere with cysteine detection. Similarly cystathionine (also with a thiol group) could affect cysteine detection. There is a factor of diminished sensitivity when testing a heterogeneous sample. As a result, those patients who do not over the top methionine metabolite levels would be mistakenly predicted with non-recurrent disease, when in fact they may have recurrent disease.

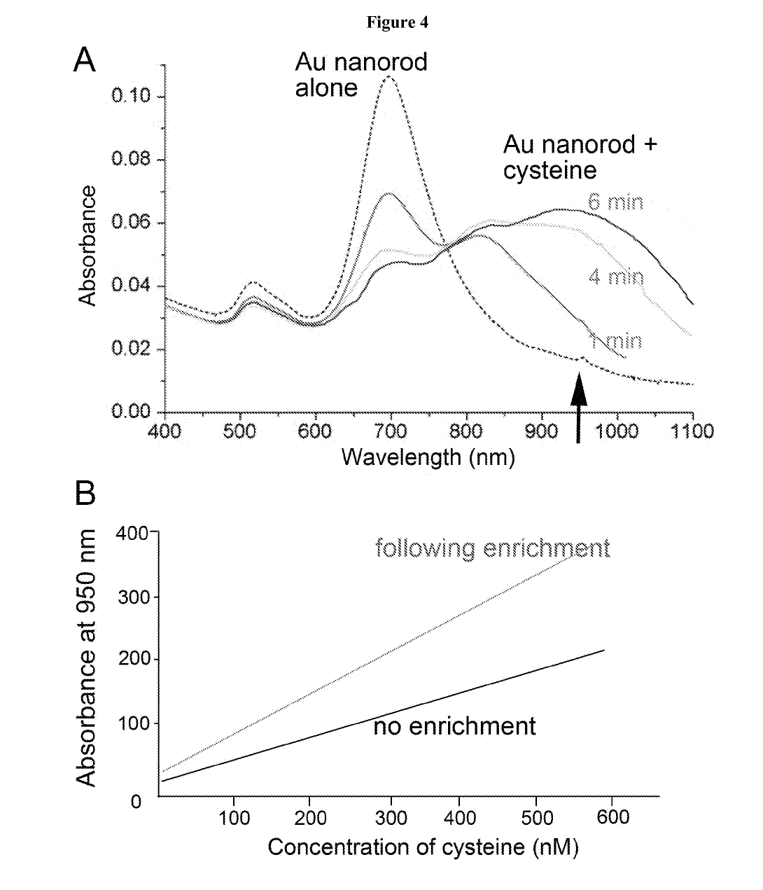

As shown in FIG. 4, this invention solves this problem both by converting both homocysteine and cystathionine to cysteine and detecting the final product cysteine. This is highly effective since we showed that homocysteine, cystathionine, and cysteine independently have strong predictive value in multi variant cox analysis including standard clinical variables of biopsy Gleason grade, serum prostate specific antigen (PSA), and clinical stage.

A typical analysis was realized by the following steps. An urine or serum sample is taken and processed with cystathionine synthase and cystathionnine lyase to convert homocysteine and cystatothionine to cysteine enzymatically in vitro. As examples, cystathionine synthase and cystathionnine lyases are cloned from the Helicobacter pylori genome. Then we detect a more pure sample that primarily contains cysteine. The processed sample was mixed with gold nanorods and is allowed to react for 10 min at room temperature. Then, the HCl solution is added into the mixture. Absorption spectrum of the reacted mixture is recorded with 1 cm path-length cell at 950 nm. Nanorods are 30 nm by 10 nm and are made of pure gold. Alternatively, nanorods can be made of selenium or cadmium with gold tips to improve detection sensitivity. Copper can be added to improve specificity.

### REFERENCES

1. Etzioni R, Penson D F, Legler J M, di Tommaso D, Boer R, et al. (2002) Overdiagnosis due to prostate-specific antigen screening: lessons from U.S. prostate cancer incidence trends. J Natl Cancer Inst 94: 981-990.
2. Andriole G L, Crawford E D, Grubb R L, 3rd, Buys S S, Chia D, et al. (2009) Mortality results from a randomized prostate-cancer screening trial. N Engl J Med 360: 1310-1319.
3. Schroder F H, Hugosson J, Roobol M J, Tammela T L, Ciatto S, et al. (2009) Screening and prostate-cancer mortality in a randomized European study. N Engl J Med 360: 1320-1328.
4. Thompson I M, Pauler D K, Goodman P J, Tangen C M, Lucia M S, et al. (2004) Prevalence of prostate cancer among men with a prostate-specific antigen level< or =4.0 ng per milliliter. N Engl J Med 350: 2239-2246.
5. Thompson I M, Ankerst D P, Chi C, Lucia M S, Goodman P J, et al. (2005) Operating characteristics of prostate-specific antigen in men with an initial PSA level of 3.0 ng/ml or lower. Jama 294: 66-70.
6. Blum D L, Koyama T, M'Koma A E, Iturregui J M, Martinez-Ferrer M, et al. (2008) Chemokine markers predict biochemical recurrence of prostate cancer following prostatectomy. Clin Cancer Res 14: 7790-7797.
7. Thompson K E, Hernandez J, Canby-Hagino E D, Troyer D, Thompson I M (2005) Prognostic features in men who died of prostate cancer. J Urol 174: 553-556; discussion 556.
8. Sreekumar A, Poisson L M, Rajendiran T M, Khan A P, Cao Q, et al. (2009) Metabolomic profiles delineate potential role for sarcosine in prostate cancer progression. Nature 457: 910-914.
9. Luka Z, Mudd S H, Wagner C (2009) Glycine N-methyltransferase and regulation of S-adenosylmethionine levels. J Biol Chem 284: 22507-22511.
10. Levy H L, Coulombe J T, Benjamin R (1984) Massachusetts Metabolic Disorders Screening Program: III. Sarcosinemia. Pediatrics 74: 509-513.
11. Allen R H, Stabler S P, Lindenbaum J (1993) Serum betaine, N,N-dimethylglycine and N-methylglycine levels in patients with cobalamin and folate deficiency and related inborn errors of metabolism. Metabolism 42: 1448-1460.
12. Allen R H, Stabler S P, Savage D G, Lindenbaum J (1993) Elevation of 2-methylcitric acid I and II levels in serum, urine, and cerebrospinal fluid of patients with cobalamin deficiency. Metabolism 42: 978-988.
13. Stabler S P, Marcell P D, Podell E R, Allen R H, Savage D G, et al. (1988) Elevation of total homocysteine in the serum of patients with cobalamin or folate deficiency detected by capillary gas chromatography-mass spectrometry. J Clin Invest 81: 466-474.
14. Horne D W, Patterson D (1988) Lactobacillus casei microbiological assay of folic acid derivatives in 96-well microtiter plates. Clinical Chemistry 34: 2357-2359.
15. Barr D B, Wilder L C, Caudill S P, Gonzalez A J, Needham L L, et al. (2005) Urinary creatinine concentrations in the U.S. population: implications for urinary biologic monitoring measurements. Environ Health Perspect 113: 192-200.
16. Pencina M J, Sr D A R, D'Agostino R B Jr (2008) Evaluating the added predictive ability of a new marker: from area under the ROC curve to reclassification and beyond. Stat Med 27: 157-172.
17. Grambsch P, Therneau T (1994) Proportional hazards tests and diagnostics based on weighted residuals. Biometrica 81: 515-526.
18. Baik H W, Russell R M (1999) Vitamin B12 deficiency in the elderly. Annu Rev Nutr 19: 357-377.
19. Roehl K A, Antenor J A, Catalona W J (2002) Serial biopsy results in prostate cancer screening study. J Urol 167: 2435-2439.
20. Capitanio U, Karakiewicz P I, Jeldres C, Briganti A, Gallina A, et al. (2009) The probability of Gleason score upgrading between biopsy and radical prostatectomy can be accurately predicted. Int J Urol 16: 526-529.
21. Beam C A, Gao W, Macias V, Liang W, Kajdacsy Balla A (2009) Sequential testing approach as an efficient and easier alternative for the validation of new predictive technologies in the clinic. J Clin Oncol 27: 1087-1090.
22. Finkelstein J D, Kyle W E, Martin J L, Pick A M (1975) Activation of cystathionine synthase by adenosylmethionine and adenosylethionine. Biochem Biophys Res Commun 66: 81-87.
23. Jentzmik F, Stephan C, Miller K, Schrader M, Erbersdobler A, et al. (2010) Sarcosine in urine after digital rectal examination fails as a marker in prostate cancer detection and identification of aggressive tumours. Eur Urol 58: 12-18; discussion 20-11.
24. Hewavitharana A K, (2010) Letter to the editor. Eur Urol 58: e39-e40.
25. Jentzmik F, Stephan C, Lein M, Miller K, Kamlage B, et al. (2011) Sarcosine in prostate cancer tissue is not a differential metabolite for prostate cancer aggressiveness and biochemical progression. J. Urol. 185:706-11.
26. Struys E A, Heijboer A C, van Moorselaar J, Jakobs C, Blankenstein M A. (2010) Serum sarcosine is not a marker for prostate cancer. Ann Clin Biochem. 47:282.
27. Cao D L, Ye D W, Zhu Y, Zhang H L, Wang Y X, Yao XD. (2011) Efforts to resolve the contradictions in early diagnosis of prostate cancer: a comparison of different algorithms of sarcosine in urine. Prostate Cancer Prostatic Dis. 14:166-72.
28. Martinez-Chantar M L, Vazquez-Chantada M, Ariz U, Martinez N, Varela M, et al. (2008) Loss of the glycine N-methyltransferase gene leads to steatosis and hepatocellular carcinoma in mice. Hepatology 47: 1191-1199.
29. Lin J, Lee I M, Song Y, Cook N R, Selhub J, et al. (2010) Plasma homocysteine and cysteine and risk of breast cancer in women. Cancer Res 70: 2397-2405.
30. Khan N, Afaq F, Mukhtar H (2010) Lifestyle as risk factor for cancer: Evidence from human studies. Cancer Lett 293: 133-143.
31. Elshorbagy A K, Nurk E, Gjesdal C G, Tell G S, Ueland P M, et al. (2008) Homocysteine, cysteine, and body composition in the Hordaland Homocysteine Study: does cysteine link amino acid and lipid metabolism? Am J Clin Nutr 88: 738-746.
32. Kane C J, Im R, Amling C L, Presti J C, Jr., Aronson W J, et al. (2010) Outcomes after radical prostatectomy among men who are candidates for active surveillance: results from the SEARCH database. Urology 76: 695-700.
33. Heady J E, Kerr S J (1975) Alteration of glycine N-methyltransferase activity in fetal, adult, and tumor tissues. Cancer Research 35: 640-643.
34. Huang Y C, Lee C M, Chen M, Chung M Y, Chang Y H, et al. (2007) Haplotypes, loss of heterozygosity, and expression levels of glycine N-methyltransferase in prostate cancer. Clin Cancer Res 13: 1412-1420.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.)

What is claimed is:

1. A system, comprising: an isolated sample from a subject, cystathionine synthase, cystathionine lyase, and a nanorod.

2. The system of claim 1, wherein the subject is human, monkey, ape, dog, cat, cow, horse, goat, pig, rabbit, mouse, or rat.

3. The system of claim 1, wherein the sample is serum, urine, blood, plasma, saliva, semen, lymph, or a combination thereof.

4. The system of claim 1, wherein the cystathionine synthase comprises a polypeptide having a sequence as set forth in SEQ ID NO: 1.

5. The system of claim 1, wherein the cystathionine lyase comprises a polypeptide having a sequence as set forth in SEQ ID NO: 2.

6. The system of claim 1, wherein the nanorod is made of gold, selenium, cadmium, copper, or a combination thereof.

7. The system of claim 1, further comprising a PSA test, clinical stage, biopsy Gleason score, pathologic Gleason score, pathologic stage, surgical margin status, lymph node involvement, or seminal vesicle involvement, or a combination thereof.

8. The system of claim 7, wherein the PSA test is a test of PSA velocity and/or total PSA level.

9. A method of predicting the probability of a recurrence of a cancer in a subject, comprising:
obtaining a sample from the subject;
processing the sample with cystathionine synthase and cystathionine lyase;
subjecting the processed sample to an assay to detect cysteine level, wherein the assay comprises a nanorod; and
predicting an increased probability of the recurrence of the cancer in the subject when the cysteine level in the subject is detected to be higher than in a non-recurrent subject.

10. The method of claim 9, wherein the recurrence is biochemical recurrence.

11. The method of claim 9, wherein the cancer is prostate cancer, colon cancer, breast cancer, lung cancer, renal cancer, or bladder cancer.
The method of claim 9, where in the sample is obtained before, during, or after cancer treatment.

12. The method of claim 9, wherein the sample is urine and the urine cysteine level in the subject is above about 210, 220, or 230 nanomoles of cysteine per milligram creatinine.

13. The method of claim 9, wherein the sample is serum and the serum cysteine level in the subject is above about 400, 410, or 420 μM of cysteine.

14. The method of claim 9, further comprising active surveillance, prostatectomy, chemotherapy, immunotherapy, hormone therapy, radiation therapy, focal therapy, systemic therapy, high frequency ultrasound (HIFU), cryo therapy, brachytherapy, or a combination thereof.

15. A method of predicting the probability of a recurrence of a cancer in a subject, comprising:
obtaining a sample from the subject;
processing the sample with cystathionine synthase and cystathionine lyase;
subjecting the processed sample to an assay to detect cysteine level, wherein the assay comprises a nanorod;
assessing at least one additional parameter; and
predicting an increased probability of the recurrence of the cancer in the subject when the cysteine level in the subject is detected to be higher than in non-recurrent subjects and/or when the additional parameter in the subject is detected to be higher or lower than in non-recurrent subjects.

16. The method of claim 15, wherein the additional parameter is PSA velocity, PSA level, pre-surgical PSA level, post-surgical PSA level, pre-treatment PSA level, post-treatment PSA level, biopsy Gleason score, clinical stage, number of positive cores, number of negative cores, Karnofsky performance status, Hemoglobin value, Lactate dehydrogenase value, Alkaline phosphatase value, Albumin level, urinary albumin level, or urinary creatinine level, or a combination thereof.

17. The method of claim 15, wherein the additional parameter is a pre-treatment parameter comprising pre-treatment PSA level, pre-treatment biopsy Gleason Score, pre-treatment clinical stage, pre-treatment urinary albumin level, or pre-treatment urinary creatinine level, or a combination thereof.

18. The method of claim 17, wherein the PSA level in the subject is above about 6.0 ng/ml in serum.

19. The method of claim 17, wherein the Gleason score in the subject is above 7.

20. A method of predicting the probability of a recurrence of a cancer in a subject, comprising:
obtaining a sample from the subject;
processing the sample with cystathionine synthase and cystathionine lyase;
subjecting the processed sample to an assay to detect cysteine level; and
predicting an increased probability of the recurrence of the cancer in the subject when the cysteine level in the subject is detected to be higher than in non-recurrent subjects.

21. A system, comprising: cystathionine synthase, cystathionine lyase, and a nanorod.

22. A method of detecting a cysteine level in a sample from a subject, comprising:
obtaining a sample from the subject;
processing the sample with cystathionine synthase and cystathionine lyase;
mixing the processed sample with a nanorod which forms a reaction mixture;
measuring a change of absorption spectrum of the reaction mixture; and
detecting the cysteine level based upon the change of absorption spectrum.
